(19) United States
(12) Patent Application Publication
(10) Pub. No.: US 2021/0212787 A1
(43) Pub. Date: Jul. 15, 2021

(54) CUSTOM-FIT MEDICAL GARMENTS AND METHODS OF MAKING THE SAME

(71) Applicant: 3D Innovations, LLC, Cape Girardeau, MO (US)

(72) Inventors: Saleem ABDULRAUF, CAPE GIRARDEAU, MO (US); NATHAN MYERS, CAPE GIRARDEAU, MO (US)

(21) Appl. No.: 17/150,237

(22) Filed: Jan. 15, 2021

Related U.S. Application Data
(60) Provisional application No. 62/961,474, filed on Jan. 15, 2020.

(51) Int. Cl. A61B 42/10; B33Y 80/00; A61B 5/107; B33Y 50/00

(52) U.S. Cl. CPC A61B 42/10; B33Y 80/00; A61B 5/1079; B33Y 50/00; A61B 5/1078

(57) ABSTRACT

A method for manufacturing a custom-fit surgical glove for a particular user includes constructing a personal three dimensional digital model of the custom-fit surgical glove using a processing computer. The method further includes translating the personal three dimensional digital model into manufacturing instructions using the processing computer, and the method includes constructing the custom-fit surgical glove using at least an additive manufacturing process and using the manufacturing instructions.

### CROSS-REFERENCE TO RELATED APPLICATIONS

This application claims the benefit of U.S. Provisional Application No. 62/961,474, filed Jan. 15, 2020, the entirety of which is hereby incorporated by reference herein.

### STATEMENT REGARDING FEDERALLY SPONSORED RESEARCH

Not Applicable.

### BACKGROUND

The present disclosure relates to custom-fit medical garments and methods of making custom-fit medical garments. More particularly, the present disclosure relates to systems and methods for taking measurements of a person (e.g., a medical professional) and using those measurements to produce a custom-fit garment using a 3D-printing technique.

Traditional medical garments, such as surgical gloves, are of one size fits all construction or come in a limited number of different sizes and/or configurations. This often results in a poor fit. A poor fit, in the example of surgical gloves, can negatively impact a surgeon's performance and/or experience in preforming procedures.

A poor fit can result from differences in a person's physiology and/or other factors. For example, finger length and thickness vary between people. Finger dimensions may also vary between hands of the same person. And, a person may have one or more fingers shaped and/or sized differently from an average which forms the basis for standard sized surgical gloves.

Given the variation between people's physiology, it is likely that a person will experience an ill-fitting medical garment in some respect. This ill-fitting garment is likely to negatively impact the function of the garment with respect to the person and the performance of a surgical or other medical procedure involving the wearing of such garment. Therefore, a custom-fit medical garment and a method of making the same is desirable.

### BRIEF SUMMARY OF THE INVENTION

Applicant has developed custom-fit medical garments and methods of making the same which greatly improve the fit of the garment for individual people. The method uses a combination of measurement techniques and additive manufacturing. This overcomes the poor fit issues of the type discussed herein.

In some embodiments, a method for manufacturing a custom-fit surgical glove for a particular user includes constructing a personal three dimensional digital model of the custom-fit surgical glove using a processing computer. The method further includes translating the personal three dimensional digital model into manufacturing instructions using the processing computer, and the method includes constructing the custom-fit surgical glove using at least an additive manufacturing process and using the manufacturing instructions.

The method can further include constructing a personal three dimensional digital model of the user's hands. The personal three dimensional digital model of the custom-fit surgical glove is constructed at least in part based on the personal three dimensional digital model of the user's hands. The personal three dimensional digital model of the custom-fit surgical glove is constructed by applying an offset to the personal three dimensional digital model of the user's hands. The method can further include generating personal measurements of the user's hands based on physiological information. The method, in that case, includes acquiring physiological information of the user, and wherein the personal measurements of the user's hands are generated based on the acquired physiological information. The acquired physiological information is received by the processing computer from a portable computing device and wherein the physiological information includes a plurality of images taken using the portable computing device. The images can include a reference object of known size. The acquired physiological information can be received by the processing computer from a three dimensional scanner and wherein the three dimensional scanner acquires the physiological information by scanning a mold or impression of the user's hands. The method can include performing additional processing on the constructed custom-fit surgical glove. The additional processing includes one or more of removal of support material, heat treating, application of one or more coatings, chemical treatment, or quality control. The additive manufacturing process can be selective laser sintering. The custom-fit surgical glove can be constructed of Latex, nitrile rubber, polyvinyl chloride, or neoprene.

In some embodiments, a system for manufacturing a custom-fit surgical glove for a particular user includes a processing computer and an additive manufacturing device. The processing computer is configured to construct a personal three dimensional digital model of the custom-fit surgical glove, and translate the personal three dimensional digital model into manufacturing instructions using the processing computer. The additive manufacturing device is configured to construct the custom-fit surgical glove using at least an additive manufacturing process and using the manufacturing instructions. The processing computer and the additive manufacturing device are in digital communication with one another, and the processing computer is configured to control the additive manufacturing device through the transmission of the manufacturing instructions to the additive manufacturing device.

The processing computer can be further configured to construct a personal three dimensional digital model of the user's hands, and wherein the personal three dimensional digital model of the custom-fit surgical glove is constructed at least in part based on the personal three dimensional digital model of the user's hands. The processing computer can be configured to construct the personal three dimensional digital model of the custom-fit surgical glove by applying an offset to the personal three dimensional digital model of the user's hands. The processing computer is configured to generate the personal three dimensional digital model of the user's hands based on physiological information. The system is configured to acquire physiological information of the user, and the processing computer is configured to generate the personal three dimensional digital model of the user's hands based on the acquired physiological information. The system can further include a portable computing device, the portable computing device being configured to take a plurality of images of the user, wherein the physiological information includes the plurality of images, and wherein the processing computer is configured to wirelessly acquire the physiological information from the portable computing device. The images can include a reference object of known size. The system can include a three dimensional scanner configured to acquire the physiological information by scanning a mold or impression of the user's hands. In such cases, the processing computer is configured to receive the physiological information from the three dimensional scanner. The additive manufacturing process can be selective laser sintering.

### DESCRIPTION OF THE DRAWINGS

In the accompanying drawings which form part of the specification:

FIG. 1 illustrates steps of a method for making a custom-fit medical garment;

FIG. 2 illustrates a system for using a handheld computing device to acquire physiological information of a person;

FIG. 3 is a perspective view of a person's hand illustrating some characteristics which may be measured;

FIG. 4 is a schematic view of a system for acquiring information about a person, processing that information, and manufacturing a custom-fit medical garment;

FIG. 5 is a schematic view of an additive manufacturing system for manufacturing a custom-fit medical garment; and

FIG. 6 is a top view of an exemplary custom-fit medical garment, in this case a surgical glove.

### DETAILED DESCRIPTION

The following detailed description illustrates the disclosed apparatuses and processes by way of example and not by way of limitation. The description enables one skilled in the art to make and use the disclosure, describes several embodiments, adaptations, variations, alternatives, and uses of the disclosure, including what is presently believed to be the best mode of making and using the apparatus. Additionally, it is to be understood that the disclosure is not limited in its application to the details of construction and the arrangements of components set forth in the following description or illustrated in the drawings. The disclosure is capable of other embodiments and of being practiced or being carried out in various ways. Also, it is to be understood that the phraseology and terminology used herein is for the purpose of description and should not be regarded as limiting.

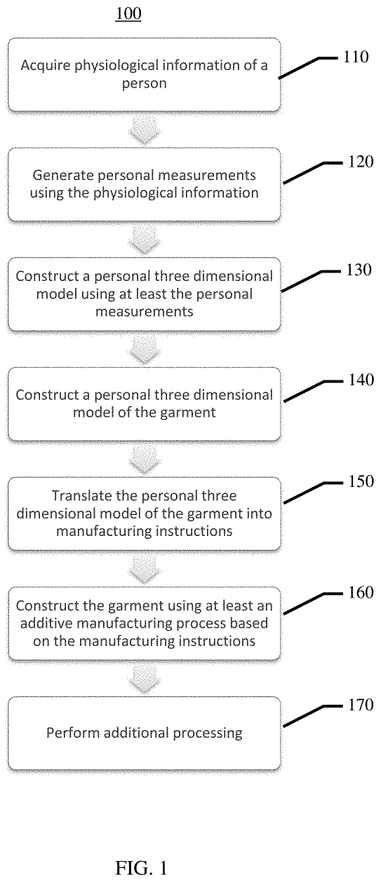

Referring generally to FIGS. 1-6, a custom-fit medical garment and a method of manufacturing the same are shown and described. Generally, measurements of a person's physiology, or relevant part thereof, are taken and this information is used to digitally model the person's physiology. The person's physiology or part thereof that is measured depends on the type of medical garment to be produced. For example, for producing a surgical glove, a person's physiology would include one or more of their hand, wrist, and/or forearm. The digital model created from the measured physiology is used to further digitally model the medical garment and generate manufacturing instructions. Those instructions are used to make the medical garment using additive manufacturing such as 3D-printing.

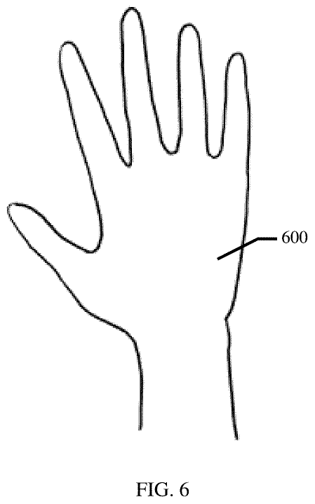

Referring specifically to FIG. 1, a method 100 is illustrated for manufacturing a custom-fit medical garment. In the illustrated embodiments, the custom-fit medical garment that is the product of the method 100 is a custom-fit surgical glove 600 (illustrated in FIG. 6). It should be understood though that the same steps of method 100 may be applied in making any custom-fit medical garment and are not limited to the manufacture of surgical gloves.

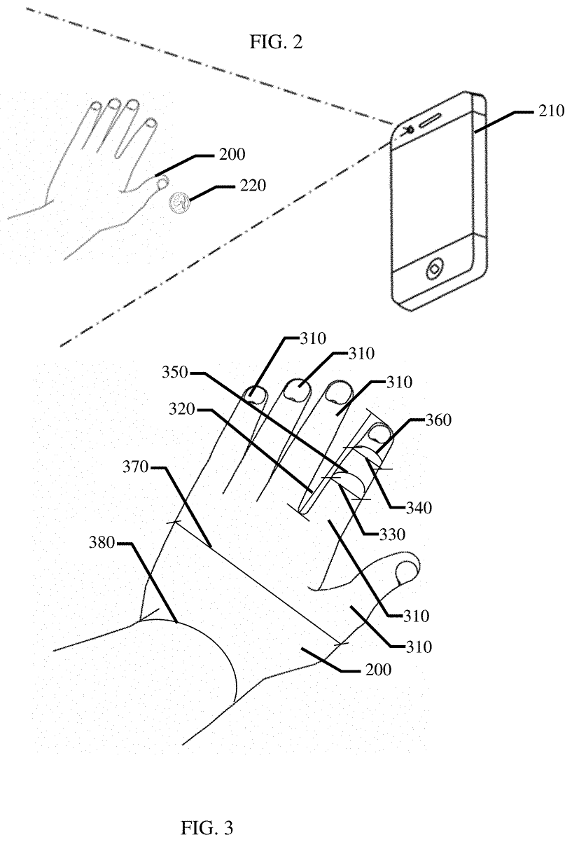

At step 110 of the method 100, physiological information of a person is acquired. This may include a variety of information gathering techniques and types of information. For example, a mold may be made of a person's hand, photographs of a person's hand may be taken, a person's hand may be laser scanned or otherwise 3D scanned, or the like. With reference to FIGS. 2-4, in the illustrated embodiment, multiple images of a person's hand 200 are taken using, for example, a smartphone 210 or other connected camera. Images are taken from a variety of angles in order to capture information for determining various measurements of the person's physiology. In one embodiment, an application running on the smartphone 210 is used to prompt a person in the views and types of images to capture. The application may also instruct the user to include a reference 220 of known dimensions in each image. For example, and without limitation, the reference 220 may be a coin, ruler, or other object of standardized size.

In an alternative embodiment, the application may use built in software to include size and geometry information with the images (e.g., in metadata). This software may use any appropriate technique known to those of skill in the art such as the techniques used in the Google Measure application. These techniques may include triangulation, shadow detection, multiple lens triangulation, detection of the change of the camera's position between multiple images based in positioning/acceleration sensors, ARCore tools, or the like.

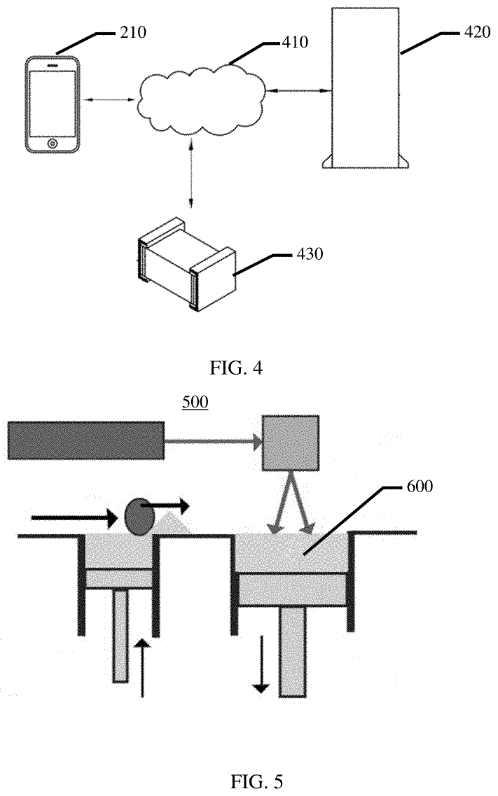

Referring to FIG. 4, the acquisition of physiological information for a person may include using a system 400 to transmit the information from the smartphone 210 using a network or cloud service 410 to a centralized processing computer 420. This information may include raw photographic information, images with metadata, measurements, images and measurements, and/or other physiological information. In alternative embodiments, a mold or impression of the person's hand may be physically shipped to the manufacturer of the custom-fit medical garment.

Referring again to FIG. 1, at step 120 of the method 100, personal measurements are generated using the physiological information. In the illustrated embodiment, the personal measurements are generated using the processing computer 420 which uses software tools and/or human input to analyze the images included in the physiological information of the person. This analysis may use any suitable technique known to a person of skill in the art including, but not limited to, using the reference 220 and a tool that measures distance in for example pixels, or other suitable software or technique (e.g., the techniques described above with respect to the application running on the smartphone 210). In an alternative embodiment, a mold of the person's physiology (e.g., their hand) may be scanned using a 3D scanning technique and the resulting scan measured using suitable techniques known to a person of skill in the art.

Referring to FIG. 3, personal measurements that are generated based on the physiological information, in the case of a surgical glove 600, may include a variety of measurements for each hand 200 (left and right) and a variety of measurements for each finger 310. For each finger 310, the measurements may include one or more of: (1) the length 320 of the finger 310 from the fingertip to the web between the finger 310 and an adjacent finger; (2) the width 330 of finger 310 at the first knuckle; (3) the width 340 of the finger 310 at the second knuckle; (4) the circumference 350 of the finger 310 at the first knuckle; (5) the circumference 360 of the finger 310 at the second knuckle; and/or (6) the height of finger 310 at one or more locations. For each hand 200, the measurements may include one or more of: (1) the width 370 of the palm at the widest point and/or at other locations; (2) the circumference 380 of the wrist; and/or the height of palm.

Referring again to FIG. 1, at step 130 of the method 100, a personal three dimensional digital model of the person's physiology is constructed using at least some of the personal measurements. The three dimensional digital model may be generated automatically and/or in part or in whole by hand. The three dimensional digital model is generated using any suitable software for three dimensional digital modeling of the type known to those skilled in the art. The personal three dimensional digital model may be generated by adjusting, based on the personal measurements, one or more initial standard digital models. The personal three dimensional digital model is constructed using the processing computer 420.

Referring again to FIG. 1, at step 140 of the method 100, a personal three dimensional digital model of the garment (e.g., the glove 600) is constructed. The personal three dimensional digital model of the garment is constructed based on the personal three dimensional digital model of the person's physiology (e.g., the personal three dimensional digital model of the person's hands). For example, the three dimensional digital model of the garment may be digital modeled using the three dimensional digital model of the person's physiology and applying an offset or tolerances to that digital model. The space occupied by the digital model of the person's physiology may be removed (e.g., to form the interior of a glove).

At step 150 of the method 100, the personal three dimensional digital model of the garment is translated into manufacturing instructions. The translation may be completed using any technique or software known to one skilled in the art. For example, software may be used to convert the digital model from a digital modeling file format such as a .dwg file type to a manufacturing file type such as a stereolithography or .stl file type suitable for use in additive manufacturing. This conversion is completed by the processing computer 420. The manufacturing instructions are suitable for use in controlling an additive manufacturing machine.

At step 160 of the method 100, the custom-fit medical garment (e.g., glove 600) is constructed using at least an additive manufacturing process and is based on the manufacturing instructions. This step may include transmitting the manufacturing instructions from the processing computer 420 to an additive manufacturing machine 430 (shown in FIG. 4). To manufacture the custom-fit medical garment, any suitable additive manufacturing process may be used including, but not limited to, selective laser sintering, fused deposition modeling, stereolithography, binder jetting, powder bed fusion, of the like. In one embodiment, selective laser sintering is used to produce the surgical glove 600. Selective laser sintering may be advantageous in that it allows for internal powder to be removed from the glove once production is complete. This and/or any other suitable technique may be used. The custom-fit medical garment (e.g., the glove 600) may be constructed of any suitable material including, but not limited to, Latex, nitrile rubber, polyvinyl chloride, neoprene, or the like.

Referring to FIG. 5, a selective laser sintering (SLS) machine 500 is illustrated according to one embodiment. The SLS machine 500 is used to produce the glove 600 (or other garment). The SLS machine selectively joins particles of material (e.g., Latex) in powder form according to the manufacturing instructions and one layer at a time. Powder may be added to the working area throughout the process as the stage is moved lower following each pass of the laser. In producing the medical garment or glove 600 further techniques may be used with the SLS machine 500 or other manufacturing equipment of the type described herein. For example, support material may be used in producing the medical garment with the support material later being removed. Alternatively, the garment, taking the glove 600 as an example, may be produced in multiple parts which are later combined. For example, the glove 600 may be produced in two halves which are later joined using any suitable technique such as heat bonding, adhesive bonding, sonic welding, or the like.

Referring again to FIG. 1, at step 170 of method 100, additional processing may be performed. This may include for example, removal of support material, touch up manufacturing using any suitable technique (e.g., heat treating, application of one or more coatings, chemical treatment, etc.), quality control, or the like. The result of method 100 is the custom-fit medical garment, e.g., the glove 600. Referring to FIG. 6, the garment, for example glove 600, is custom-fit to provide for a better fit to the person than standardly available sizes. This may provide several advantages including, but not limited to, improved tactile feel for a surgeon or doctor performing an exam procedure.

Changes can be made in the above constructions without departing from the scope of the disclosure. It is intended that all matter contained in the above description or shown in the accompanying drawings shall be interpreted as illustrative and not in a limiting sense.

We claim:

1. A method for manufacturing a custom-fit surgical glove for a particular user, the method comprising:
constructing a personal three dimensional digital model of the custom-fit surgical glove using a processing computer;
translating the personal three dimensional digital model into manufacturing instructions using the processing computer; and
constructing the custom-fit surgical glove using at least an additive manufacturing process and using the manufacturing instructions.

2. A method in accordance with claim 1 further comprising constructing a personal three dimensional digital model of the user's hands, and wherein the personal three dimensional digital model of the custom-fit surgical glove is constructed at least in part based on the personal three dimensional digital model of the user's hands.

3. A method in accordance with claim 1 wherein the personal three dimensional digital model of the custom-fit surgical glove is constructed by applying an offset to the personal three dimensional digital model of the user's hands.

4. A method in accordance with the method of claim 1 further comprising generating personal measurements of the user's hands based on physiological information.

5. A method in accordance with claim 4 further comprising acquiring physiological information of the user, and wherein the personal measurements of the user's hands are generated based on the acquired physiological information.

6. A method in accordance with claim 5 wherein the acquired physiological information is received by the processing computer from a portable computing device and wherein the physiological information includes a plurality of images taken using the portable computing device.

7. A method in accordance with claim 6 wherein the images include a reference object of known size.

8. A method in accordance with claim 5 wherein the acquired physiological information is received by the processing computer from a three dimensional scanner and wherein the three dimensional scanner acquires the physiological information by scanning a mold or impression of the user's hands.

9. A method in accordance claim 1 further comprising performing additional processing on the constructed custom-fit surgical glove, wherein the additional processing includes one or more of removal of support material, heat treating, application of one or more coatings, chemical treatment, or quality control.

10. A method in accordance with claim 1 wherein the additive manufacturing process is selective laser sintering.

11. A method in accordance with claim 1 wherein the custom-fit surgical glove is constructed of Latex, nitrile rubber, polyvinyl chloride, or neoprene.

12. A system for manufacturing a custom-fit surgical glove for a particular user, the system comprising:
a processing computer configured to construct a personal three dimensional digital model of the custom-fit surgical glove, and translate the personal three dimensional digital model into manufacturing instructions using the processing computer; and
an additive manufacturing device configured to construct the custom-fit surgical glove using at least an additive manufacturing process and using the manufacturing instructions,
wherein the processing computer and the additive manufacturing device are in digital communication with one another, and wherein the processing computer is configured to control the additive manufacturing device through the transmission of the manufacturing instructions to the additive manufacturing device.

13. A system in accordance with claim 12, wherein the processing computer is further configured to construct a personal three dimensional digital model of the user's hands, and wherein the personal three dimensional digital model of the custom-fit surgical glove is constructed at least in part based on the personal three dimensional digital model of the user's hands.

14. A system in accordance with claim 13 wherein the processing computer is configured to construct the personal three dimensional digital model of the custom-fit surgical glove by applying an offset to the personal three dimensional digital model of the user's hands.

15. A system in accordance with claim 13, wherein the processing computer is configured to generate the personal three dimensional digital model of the user's hands based on physiological information.

16. A system in accordance with claim 15, wherein the system is configured to acquire physiological information of the user, and wherein the processing computer is configured to generate the personal three dimensional digital model of the user's hands based on the acquired physiological information.

17. A system in accordance with claim 16, further comprising a portable computing device, the portable computing device configured to take a plurality of images of the user, wherein the physiological information includes the plurality of images, and wherein the processing computer is configured to wirelessly acquire the physiological information from the portable computing device.

18. A system in accordance with claim 17 wherein the images include a reference object of known size.

19. A system in accordance with claim 17, further comprising a three dimensional scanner configured to acquire the physiological information by scanning a mold or impression of the user's hands, and wherein the processing computer is configured to receive the physiological information from the three dimensional scanner.

20. A system in accordance with claim 12 wherein the additive manufacturing process is selective laser sintering.
